SEQUENCE LISTING (6 sequences)

SEQ ID NO: 1 - protein, 309 aa, Plasmodium falciparum
  PfFNT_PF3D7_0316600_PlasmoDB
MPPNNSKYVLDPVSIKSVCGGEESYIRCVEYGKKKAHYSNLNLLAKAILAGMFVGLCAHASGIAGGLFYYHKLREIVGASMSVFVYGFTFPIAFMCIICTGSDLFTGNTLAVTMALYEKKVKLLDYLRVMTISLFGNYVGAVSFAFFVSYLSGAFTNVHAVEKNHFFQFLNDIAEKKVHHTFVECVSLAVGCNIFVCLAVYFVLTLKDGAGYVFSVFFAVYAFAIAGYEHIIANIYTLNIALMVNTKITVYQAYIKNLLPTLLGNYIAGAIVLGLPLYFIYKEHYYNFERSKRDNNDAQMKSLSIELRN

SEQ ID NO: 2 - protein, 412 aa, Toxoplasma gondii
  TgFNT1_TGGT1_209800_ToxoDB
MVVTASPDTYLHVIDYGLKKVRLRFDRLLLQAFMAGVYIGMAGNACISLAGGFSTDPADPKAITAGVQKFIYASIFPVAFIAIIMTGAELFTGNTMTMLICWFERRITIWQLLQNWAGSFLGNWLGTMFSAYFLTYLCCPFDHDPYLSYLNYTAASKVSYGWGSCFLRGVGCNTWVCLAVWFVVACDDAAGKILALWFPIVAFVLSSYEHIIANLYTLQLCAMLGVDTSLADMIAFNLLPTLLGNLFGGCGLIGMVYFYNFYPVVGHGDDAAEGSICGSSEKEECPSLVGVPRGASVNSLAVSAIPSVFSAPRGQRESFAGESSTLVMGDIKRQRSMASTRKLGGGADKKDVQLTVRQFDETEMQSTMEDMFGLEDPRNAPKGNPGTNPPSKSPESSATGGTGAAASPTATS

SEQ ID NO: 3 - protein, 463 aa, Toxoplasma gondii
  TgFNT3_TGGT1_292110_ToxoDB
MCSIPPLRLLEVVDDLTCLVNSSQYSRWDFFTFTLPGSTLPMRKEFTSSCKGSFSNHPVPDHPCKLVVFVRPKMVVTAGADAYLKILEYGVKKTQLRIDRLLLQAFMAGIFVAMAGHCCTVLAGSYPTDPGDPLAVAKPTQKFIYGALFPVAFICIILTGAELFTGNTMTMLICYFQKRVTMLQLGVNWLGSLAGNWLGALFGAYFLSYLTGALGDEHVRQFLFRTCVNKISYGWGECFLRGVGCNTFVCLAVWAVIASENVAGKVLVMWFPIVAFCVGGYEHIIANMYTLQAGLMAGAPVAILDVIAFNFLPTLLGNIVGGCLLVGAVYAYNFYPTLSYTETTGAKVYVQEVGPVLDRRSSMQVSMTEREPDGQVVTEYEAVPFESFGGEYIVNKHATMAAPIPSRASSFLYPFQWQRQRSQSGNLSTHARLDLPNRPVEPPSDGLEVTPQSQTAESVAQQV

SEQ ID NO: 4 - protein, 501 aa, Toxoplasma gondii
  TgFNT2_TGGT1_229170_ToxoDB
MVLAASPEAYRKVIEYGIKKTKLRIDRLFLQAIMAGIYVGMAGHACTALAGAYSTDPANPLAVSKATQKFLYASLFPVAFIAIIFTGAELFTGNTMTMLVCLLERRVTALQLCINWICSLVGNWAGALFAAYFLSYLPGVLQDPDHLHYLEDVAAHKTELSFLQCFCLAVGCNTFVCLAVWFVIASDDAAGKIMSMWFPIVSFCVAGYEHIIANFYTLQCALMHGVGPGVGTVILKNFIPTLLGNIVGGCGLVGAVYWYNFYPTVCVVQEARQPLPLSENAPSSTRQVVADLFSLWGRESSTPGVSASPPDAATNAGCSALDPPRNALLAAGKNFGNLSAGDRGALAEGIPGGACEDCLLVPRASFGGEYHPPQQGDAGRWCKPSKAAVGSGGVLCHVQSPAALEAVSNSPLRENSGVPSGGLLLCEGRVRRSSREREPERGGEEEEGASPEEEHPAVTLSIPPTDFHPHVPREVEQSSLLEETRVAAENSALEEHPASTI

SEQ ID NO: 5 - protein, 356 aa, Entamoeba histolytica
  EhFNT_EHI_198990_AmoebaDB
MPREKPRADEIAIEMMSVCEDETEVEQDPRELYEEEMKEQQQIDCSKQQKEVVAIEELEKRNINKHFFSIQPNTQIPVISSNYIAPVDTSRLLVLIGKTKATYPIMKMFSLSVLAGMLLSVGGLLSITIGKGIPSSDIGIQKIVFGFFNSVGLNLVVLCGGELFTSNCAFLIPGFMEGAYSRWLFFKTHFVVYFGNLVGSIFVSTYFGKLLGSFESPMYLSAVKQIGETKVAMNWGRALLSGIGCNWLVCCAVYFSASAKDLLSKLVVISFLVLTFASLEFENCVGNMFLLSLSHMYGGNFTLGQWILNNLIPVSIGNFIGGTFLLGIPLWYVHVSNVYNIPFLDPLYQQSQAKTQ

SEQ ID NO: 6 - protein, 269 aa, Salmonella enterica subsp. enterica serovar Typhimurium
  StNirC_AAA27040_NCBI
MFTDSINKCAAKLRASAPVSANNPLGFWVSSAMAGAYVGLGIILIFTLGNLLDPSVRPLVMGATFGIALTLVIIAGSELFTGHTMFLTLGVKAGTISHGQMWAILPQTWLGNLVGSVFVALLYSWGGGSLLPVDTSIVHSVALAKTTAPATVLFFKGALCNWLVCLAIWMAIRTEGTAKFLAIWWCLLAFIASGYEHSVANMTLFALSWFGHHSDAYTLAGIGHNLLWVTLGNTLSGVVFMGLGYWYATPKSERPAPAKINQPEAAANN

(19) United States
(12) Patent Application Publication
(10) Pub. No.: US 2019/0358174 A1
(43) Pub. Date: Nov. 28, 2019

(54) ACTIVE AGENTS AGAINST PROTOZOA

(71) Applicant: Christian-Albrechts-Universität zu Kiel, Kiel (DE)

(72) Inventors: Eric Beitz, Dänischenhagen (DE); André Golldack, Kiel (DE); Björn Henke, Kiel (DE)

(21) Appl. No.: 16/319,619

(22) PCT Filed: Jun. 8, 2017
(86) PCT No.: PCT/EP2017/064026

§ 371 Date: Jan. 22, 2019

(30) Foreign Application Priority Data
Jun. 9, 2016 (EP) 16173828.1

(51) Int. Cl. A61K 31/035; A61K 31/122; A61K 31/40; A61K 31/343

(52) U.S. Cl. CPC A61K 31/035; A61K 31/343; A61K 31/40; A61K 31/122

(57) ABSTRACT

The invention relates to compounds, which are directed against protozoa, for use in the treatment of protozoal infections. The compounds are characterized in that they inhibit the formate-nitrite transporter of protozoa.

The present invention relates to compounds which are directed against protozoa and especially to compounds for use in the treatment of infections by protozoa. Furthermore, the invention relates to a process for inhibition, preferably for destruction, of protozoa by contacting of protozoa with at least one of the compounds. The compounds are characterized in that they inhibit the formiate-nitrite-transporter of protozoa.

### STATE OF THE ART

EP 2483274 B1 describes a multitude of active agents against malaria which shall inhibit the dihydroorotate dehydrogenase of plasmodium.

EP 2526090 B1 describes aminopyridine derivatives as a pharmaceutical active agent, especially against malaria.

The organisation Medicines for Malaria Venture (MMV), under a total of approximately 400 other compounds describes the following as active agents against malaria:

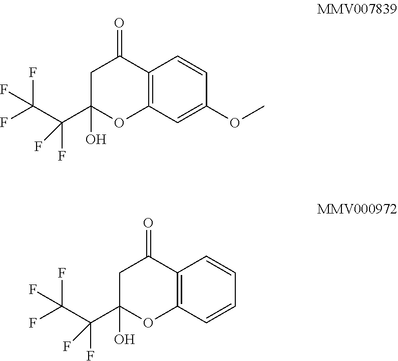 [SMILES: COc1ccc2c(c1)OC(O)(C(F)(F)C(F)(F)F)CC2=O ; O=C1CC(O)(C(F)(F)C(F)(F)F)Oc2ccccc21]

### OBJECT OF THE INVENTION

The object of the invention is to provide alternative compounds which are active as active agents, especially against protozoa. Preferably the alternative compounds are active against another target molecule of protozoa than hitherto known.

### DESCRIPTION OF THE INVENTION

The invention achieves the object by the features of the claims, especially by means of a compound and derivatives thereof for use against protozoa, respectively for treatment of an infection by protozoa, wherein the compound has the following structure I or consists thereof, in which the radical R1 is a perfluoroalkyl, in which the alkyl is a straight-chain or branched C₁- to C₄-alkyl:

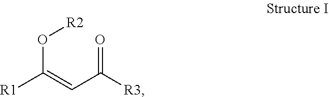

wherein
R1=perfluoro-C₁- to C₄-alkyl, straight-chain or branched, especially trifluoromethyl, pentafluoroethyl, heptafluoropropyl, nonafluorobutyl, of these preferably pentafluoroethyl, including solvates and salts of these.
R2 is H or a C₁- to C₁₂-alkyl, e.g. methyl, ethyl, n-propyl, n-butyl, n-pentyl, n-hexyl, n-octyl, n-decyl, n-dodecyl, iso-propyl, iso-butyl, tert-butyl, 2,2-dimethylpropyl or cyclohexyl, of these preferably H or ethyl, or R2 is a carbonyl group with H or a C₁- to C₁₂-alkyl, which is e.g. methyl, ethyl, n-propyl, n-butyl, n-pentyl, n-hexyl, n-octyl, n-decyl, n-dodecyl, iso-propyl, iso-butyl, tert-butyl, 2,2-dimethylpropyl or cyclohexyl. On the basis of current analyses it is assumed that the active pharmacophore is formed by structure I with R1.

Preferably, R3 is an aromatic compound, preferably an aromatic 5-ring or an aromatic 6-ring, which can optionally have aromatic rings condensed to it. The aromatic compound can be bound directly or via a C₁- to C₃-alkyl. A hydroxyl group in R3 in a distance of two carbon atoms to the carbonyl-carbon of structure I results in the formation of a hemiketal like in MMV007839; the hemiketal represents a well membrane-permeable internal prodrug. R3 is e.g. selected from the group which comprises or consists of aromatic compounds, which are substituted in para- or meta-position to structure I with a straight-chain or branched C₁- to C₁₂-alkyl radical, C₁- to C₁₂-alkoxy radical or halogen, especially the following radicals

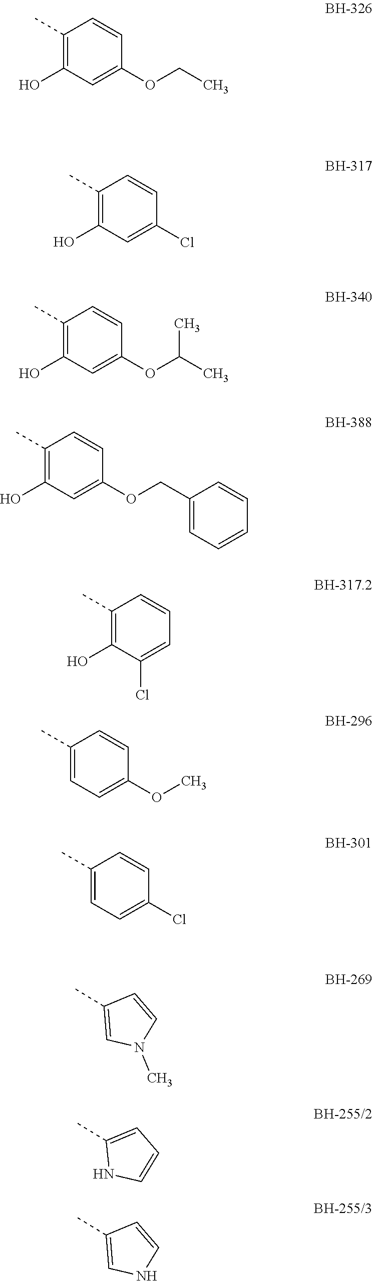

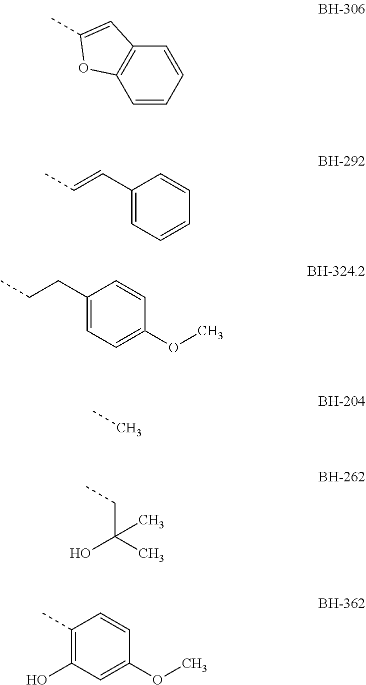

The designations under the radicals R3 give the names of the compounds from structure I wherein R1=perfluoro-C₁- to C₄-alkyl, preferably pentafluoroethyl, R2=H or ethyl, preferably H and the radical given for R3.

The aromatic compound can be a phenyl, a pyrrolyl, furyl, pyridyl or benzofuryl or the like. Preferably, R3 has a phenyl ring, which at a spacing of two carbon atoms from the carbonyl-C-atom of structure I, correspondingly in ortho-position to the keto group, has a hydroxyl group, and in para-position to the keto group has a halogen atom, especially Cl, or an alkoxy radical, e.g. a methoxy group, preferably an ethoxy group or a propoxy group, or consists thereof. The propoxy group can be an n-propoxy group or an isopropoxy group.

Preferred compounds, in which R3 is a phenyl radical which has a hydroxyl group in ortho-position to the keto group and has a Cl or an ethoxy group or a propoxy group in para-position to the keto group, are e.g. the compounds BH-326, BH-317 and BH-340.

According to the invention, the open chain vinylogous carbon acid is the active, substrate-like effective form, whereas the hemiketals MMV007839 and MMV0972 are reversible, internal prodrugs having a good resorption in the malaria parasite. The use of the prodrug-principle by introduction of a hydroxyl group in R3 can therefore increase the availability in the parasite.

Optionally, the compound MMV007839 and/or the compound MMV000972 and/or the open chain vinylogous carbonyl compound which is in equilibrium with the compound MMV007839 and/or the compound MMV000972, or their isomers, respectively hemiketal isomers, are exempt from the compounds of structure I.

These compounds are characterized in that they inhibit the formiate-nitrite-transporter protein (FNT) from protozoa. Humans do not posses this formiate-nitrite-transporter proteins. The human lactate transporter is inhibited significantly less by the compounds. The inhibition of FNT results in the protozoa not effectively transporting acetate and lactate which is generated from pyruvate e.g. by the anaerobic metabolism of glucose so that the cells over-acidify and die.

Generally, only protozoa against which the compounds are used as an active agent have the formiate-nitrite-transporter proteins (FNT). Preferred protozoa for application of the compounds are single cell parasites of humans and animals, especially Plasmodium falciparum (Pf, PfFNT, Sequence-ID PF3D7_0316600 PlasmoDB) and Plasmodium vivax (Pv; PvFNT, Sequence-ID PVX_095405 PlasmoDB), two causative agents of malaria, Toxoplasma gondii (Tg; TgFNT1, Sequence-ID TGGT1_209800 ToxoDB; TgFNT2, Sequence-ID TGGT1_292110 ToxoDB; TgFNT3, Sequence-ID TGGT1_229170 ToxoDB), the causative agent of toxoplasmosis, Entamoeba histolytica (Eh; EhFNT, Sequence-ID EHI_198990 AmoebaDB), the causative agent of the amoebic dysentry.

The invention is now described by way of examples with greater detail.

### Example 1: Effect of Compounds Against FNT

The effect of the following compounds on the growth of Plasmodium falciparum was tested using cultivated plasmodia:

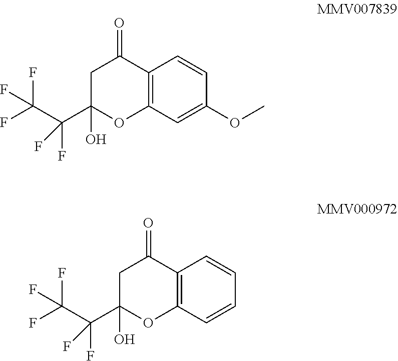 [SMILES: COc1ccc2c(c1)OC(O)(C(F)(F)C(F)(F)F)CC2=O ; O=C1CC(O)(C(F)(F)C(F)(F)F)Oc2ccccc21]

The inhibitory effect IC50 on the growth of the plasmodium culture was determined to 140 nM for MMV007839, to 1.7 μM for MMV000972. The plasmodium culture was maintained in 5% 0+ erythrocytes in RPMI 1640 medium with 0.5% Albumax at 37° C. The IC50 was determined 48 h following addition of dilutions of the compounds by way of counting parasitaemia by FACS. A change of medium was made after 24 h and the compounds were added freshly.

Upon treatment of the cultivated plasmodia with MMV007839 at a concentration corresponding to the threefold IC50-concentration, resistant plasmodia were obtained, in which the IC50 had shifted to 35 μM. Sequencing of the FNT-gene of the resistant plasmodia showed a mutation Gly107Ser on the protein level.

Except for the compound BH-296 no compound is known to-date, which in a pharmaceutically acceptable concentration range is also effective against the resistant parasites described in Example 1.

The FNT of the wild-type plasmodium (available under accession number PF3D7_0316600 in the data bank PlasmoDB) and of the resistant plasmodia (position of the mutation in the open reading frame: guanine 319 to adenine) were expressed in the yeast Saccharomyces cerevisiae W303-1A jen1Δ ady2Δ from plasmid pDR196 (PMA promoter). In the yeast, the endogenous genes for the monocarboxylate transporter Jenlp (sequence accession No. CAA82062 NCBI) and Ady2p (sequence accession No. KZV 12856) were deleted (obtained from M. Casal, Universidade do Minho, Portugal).

This yeast was grown in SD medium with addition of adenine, histidine, leucine, tryptophan and 2% (wt/v) glucose at 30° C. up to an OD₆₀₀ of 0.8 to 1.0, harvested by centrifugation and washed once with water and centrifuged down again, and suspended in 50 mM HEPES/TRIS (pH 6.8) and adjusted to an OD₆₀₀ of 50±10% and stored on ice. For contacting with one of the compounds a compound of one step of a dilution series in DMSO was provided in a reaction vessel and onto this 80 μL of the yeast suspension were pipetted. After an incubation on ice for 15 to 20 min 20 μL 5 mM Na-L-lacate plus 0.04 μCi radioactively labelled L-(1-¹⁴C) lactate was added. The lactate concentration obtained was 1 mM. After an incubation of 30 s the uptake of lacatate was stopped by a rapid dilution by means of adding of 1 ml ice cold water. From the yeast suspension diluted this way the yeast cells were brought onto a filter membrane by vacuum filtration, washed with 7 ml cold water and transferred with the filter membrane into 3 ml scintillation cocktail. After 24 h of incubation at 18° C. in the scintillation cocktail, in which the cells were lysed, the amount of radioactively labelled lactate was measured by means of a scintillation counter. The total amount of lactate which was taken up by the yeast was calculated from the measured amount of radioactively labelled lactate. As a positive control the yeast was treated equally in parallel testing with DMSO without one of the compounds and was regarded as 100% FNT activity, respectively 0% inhibition. In testings with the compound a lower amount of lactate taken up was found in comparison to the parallel positive control. As a negative control yeast was treated in parallel in which the endogenous genes for the monocarboxylate transporters Jenlp and Ady2p were deleted, but the FNT from plasmodium was not expressed. This negative control was regarded as 0% FNT activity, respectively 100% inhibition.

The IC50 values determined using the yeast for the wild-type FNT and for the FNT mutant showed the same shift as the IC50 values of the plasmodium culture. On the one hand, this shows that the tested compounds present their effect onto plasmodium by interaction with the FNT, respectively that FNT is the target molecule of these compounds in the inhibition of plasmodium. On the other hand this result shows that the yeast expressing the FNT from plasmodium can be used as a representative for plasmodium itself in processes for analysis of the inhibitory effect of compounds onto plasmodium. The formation of the hemiketal prodrug form has a general positive effect onto the effect in a plasmodium culture, while the yeast takes up the open chain form equally well.

### Example 2: Production of Compounds

The production of the basic body of the compounds can occur by synthesis using the following steps:

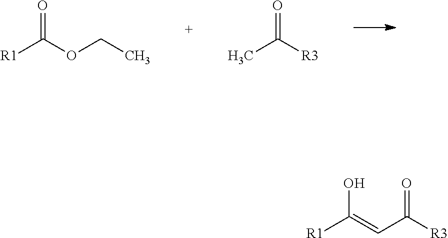

wherein R=perfluoro-C₁- to C₄-alkyl, R3 is one of

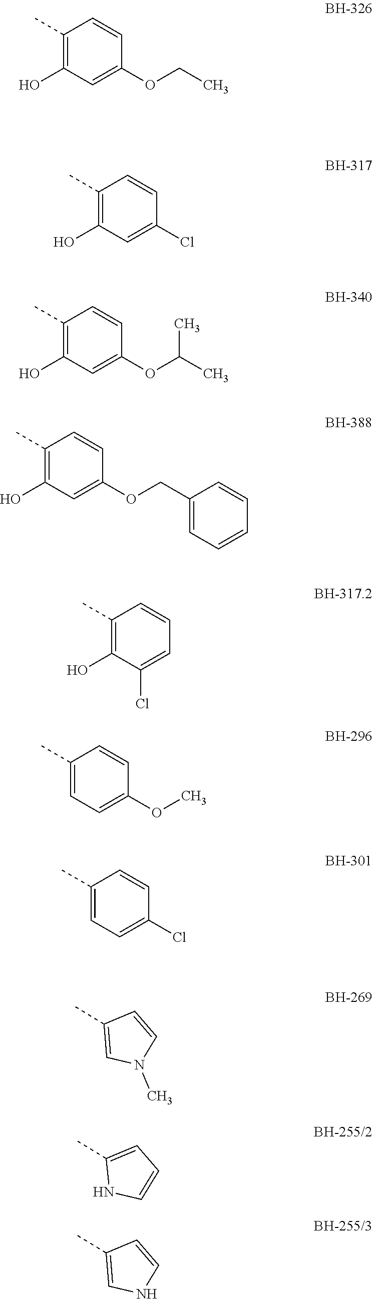

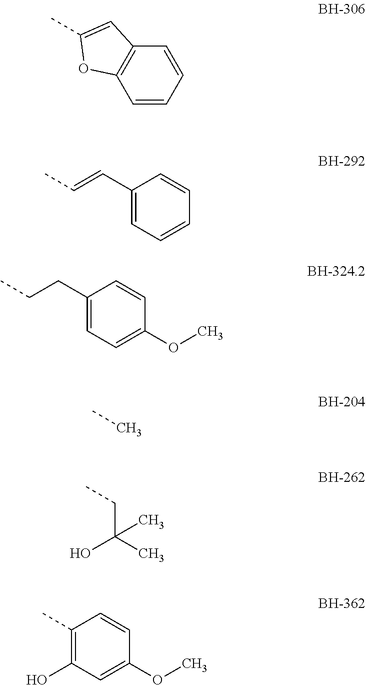

For the synthesis, 7.5 ml water-free tetrahydrofuran (THF) and 0.34 g (42.8 mmol) finely dispersed lithium hydride are provided in a dried 100 ml three-neck flask, provided with a reflux cooler and a dropping funnel, stirred and heated to boiling. To this suspension, a mixture of 15.0 mmol perfluoro alkyl carbon acid-ethyl ester, the perfluoro alkyl group of which corresponds to R1, and 12.5 mmol of the keton substituted with R3, dissolved in dried THF, is slowly added by drops. The reaction mixture is boiled for 3 h under reflux. Subsequently, THF is removed at the rotary evaporator and the residue obtained is mixed with 60 ml of a cold mixture of acetic acid and water (7:50) and the mixture is extracted with 2×100 ml ethyl acetate. The combined phases are dried over sodium sulfate, rotary concentrated, purified by column chromatography over silica gel (cyclohexane/ethyl acetate 90:10) and re-crystallized in n-hexane. The structure of the compounds is confirmed by means of 1H-NMR, 13C-NMR, 19F-NMR and mass spectrometry (LC-MS ESI). The esterification of the vinologous carbon acid according to

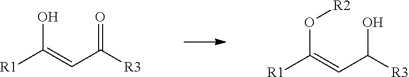

occurred by providing 5.0 ml water free dimethyl formamide (DMF) with 2 mmol of the vinologous carbon acid compound and 2 mmol (652 mg) cesium carbonate in a dry 100 ml three-neck flask, equipped with reflux cooler and dropping funnel, and stirring for 1 h at 70° C. 2.2 mmol (442 mg) p-toluene sulfonic acid ethyl ester, dissolved in 5 ml dried DMF, were slowly added dropwise to the suspension and stirred for 6 h at 70° C. The mixture was given onto water, extracted 2× with diethyl ether and the organic phases were dried over sodium sulfate.

Following concentration of the ether on the rotary evaporator the cooled product obtained was purified by column chromatography over silica gel (cyclohexane/ethyl acetate/diethylamine 94:5:1). The structure of the compounds was confirmed by means of 1H-NMR, 13C-NMR, 19F-NMR and mass spectrometry (LC-MS ESI).

### Example 3: Effect of Compounds Against FNT

As a representative for a Formiate-Nitrit-Transporter protein (FNT) from protozoa, the FNT of Plasmodium falciparum as wild-type and as Gly107Se mutant was used, which were each expressed in yeast and incubated with dilutions of the compounds to be tested as described in Example 1.

Therein, the compounds

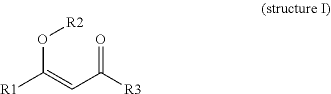

with
R1=pentafluoro ethyl or heptafluoro propyl (see BH-362), R2=H and
R3 one of the radicals

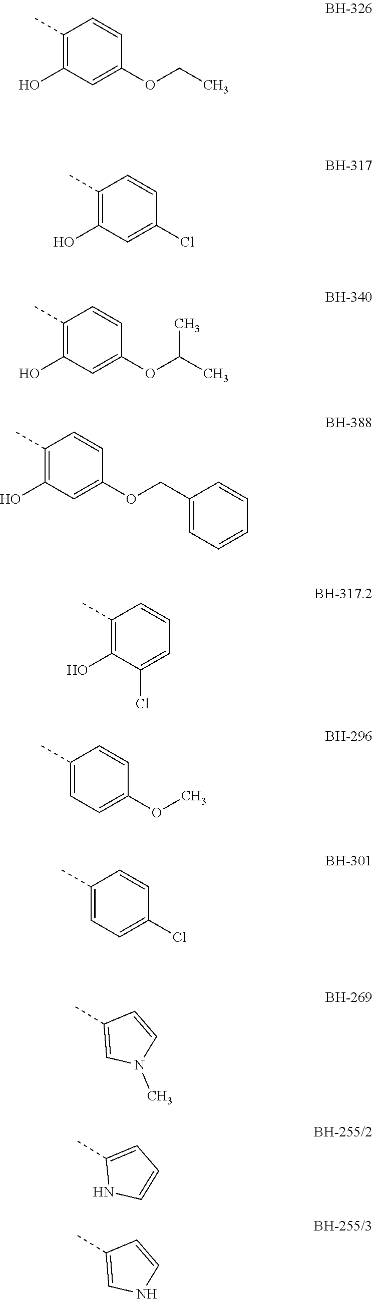

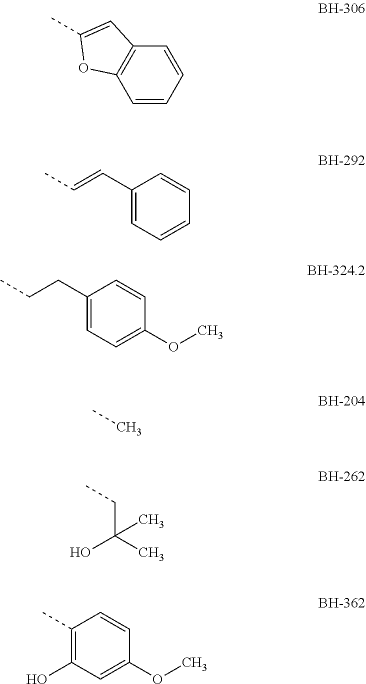

were utilized, which are each designated in accordance with the designation given under R3.

The following inhibition values were obtained:

| | yeast test system | | | |
|---|---|---|---|---|
| | | IC50 against | plasmodium culture | |
| | IC50 against | Gly107Ser | | IC50 against |
| | FNT wild-type | mutant of FNT | IC50 against | Gly107Ser |
| | (μM) | (μM) | wild-type (μm) | mutant (μM) |
| R1 = penta- | | | | |
| fluoro ethyl; | | | | |
| R2 = H | | | | |
| BH-326 | 0.24 | not determined | 0.05 | not determined |
| BH-317 | 0.12 | not determined | 0.37 | not determined |
| BH-340 | 0.40 | not determined | 0.10 | not determined |
| BH-388 | 0.89 | not determined | not determined | not determined |
| BH-317.2 | 0.25 | not determined | not determined | not determined |
| BH-296 | 0.14 | 2.7 | 3.4 | 8.9 |
| BH-301 | 0.12 | 5.8 | not determined | not determined |
| BH-269 | 0.13 | 15 | not determined | not determined |
| BH-255/2 | 0.16 | 12 | not determined | not determined |
| BH-255/3 | 0.20 | 13 | not determined | not determined |
| BH-306 | 0.18 | 15 | not determined | not determined |
| BH-292 | 0.26 | 25 | not determined | not determined |
| BH-324.2 | 0.16 | 6.3 | not determined | not determined |
| BH-204 | 1.9 | 8.1 | not determined | not determined |
| BH-262 | 1.1 | 490 | 10 | 70 |
| R1 = hepta- | | | | |
| fluoro propyl; | | | | |
| R2 = H | | | | |
| BH-362 | 0.25 | not determined | 0.57 | not determined |

The IC50 values show that the tested new compounds significantly inhibit the FNT of plasmodium. From these results it is also evident that especially BH-326, BH-340 and BH-317 both effect an effective inhibition of the FNT as shown in the yeast test system, and an effective inhibition of the plasmodium culture. Therein, these compounds have a phenyl group as R3 which in ortho-position to the keto group has a hydroxyl group and in BH-317 in para-position to the keto group has a Cl halogen atom, respectively BH-326 and BH-340 have an alkoxy radical in para-position to the keto group, wherein the alkoxy radical of BH-326 is an ethoxy group and in BH-340 is an isopropoxy group.

Further the IC50 values show that preferably the compounds BH-296, BH-301 and BH-324.2 also inhibit the Gly107Ser mutant significantly.

The compounds show a dependency of the IC50 from the time of preincubation, before lactate is added to the cells. Therein it shows that a longer preincubation results in an increased inhibition of transport. Upon prolongation of the preincubation with MMV007839 to 24 h prior to addition of lactate an IC50 of 15 nM is determined instead of the IC50 of 170 nM after 20 min. The inhibition of FNT could not be reduced by washing of the cells with 50 mM HEPES-Tris, pH 6.8, and the inhibition was maintained for hours. It is therefore assumed that this compound in any case is not removed from FNT by washing. This result indicates that the compounds bind irreversibly to FNT, respectively that the compounds result in a continued blockage of FNT by suicide inhibition. It is assumed that the compounds imitate the structure of two lactate molecules, one in the anionic form (vinylogous carbon acid of structure I) and one in the neutral lactic acid form (fluoro alkyl radical, R1).

Therefore it is presently assumed that the pharmakophor of structure I is formed with the perfluoro-C₁ to C₄-alkyl as R1 and that the radical R3 can be varied, e.g. can significantly be sterically enlarged over an aromatic radical R3. The fluoroalkyl R1 can project into the lipophilic transport channel of the FNT, while the negatively charged structure I electrostatically interacts with the transporter entrance of the FNT.

1. Compound for use in the treatment of infections by protozoa, which has the structure I, in which the radical R1 is a perfluoro alkyl, in which the alkyl is a straight-chain or branched C₁- to C₄-alkyl

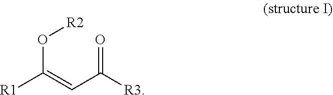

2. Compound according to claim 1, wherein the protozoa have a formiate-nitrite-transporter protein (FNT).

3. Compound according to claim 1 wherein the use is for treatment of an infection of a human or an animal by protozoa.

4. Compound according to claim 1, wherein R2 is H or a C₁- to C₁₂-alkyl or R2 is a carbonyl group with H or a C₁- to C₁₂-alkyl.

5. Compound according to claim 4, wherein R1 is trifluoro methyl, pentafluoro ethyl, heptafluoro propyl or nonafluoro butyl.

6. Compound according to wherein R3 is an aromate, which is bound directly or by a C₁- to C₃-alkyl and which in para position or meta position to structure I is substituted with a straight-chain or branched C₁- to C₁₂-alkyl radical or a C₁- to C₁₂-alkoxy radical or a halogen.

7. Compound according to claim 4, wherein the C₁- to C₁₂-alkyl of R2 and/or C₁- to C₁₂-alkyl radical of R3 independent from one another is methyl, ethyl, n-propyl, n-butyl, n-pentyl, n-hexyl, n-octyl, n-decyl, n-dodecyl, iso-propyl, iso-butyl, tert-butyl, 2,2-dimethylpropyl or cyclohexyl.

8. Compound according to claim 1 wherein R3 contains a hydroxyl group in a spacing of two carbon atoms from the carbonyl-C-atom of structure I for formation of the hemiketal prodrug.

9. Compound according to claim 1, wherein R3 has a phenyl ring which in ortho-position to the carbonyl-C-atom has a hydroxyl group and in para position to the carbonyl-C-atom has an alkoxy radical.

10. Compound according to claim 8, wherein the alkoxy radical is a methoxy group, an ethoxy group or a propoxy group.

11. Compound according to claim 1, wherein R3 is selected among

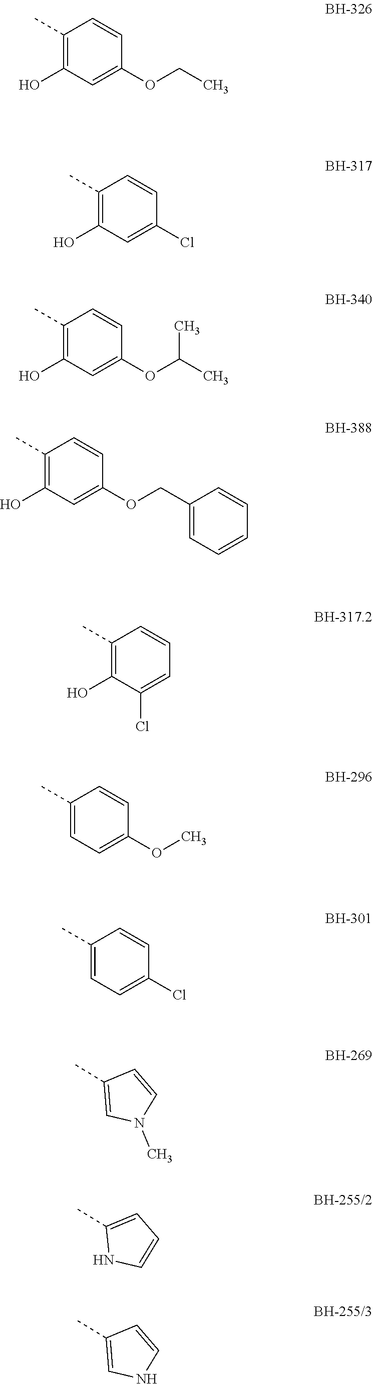

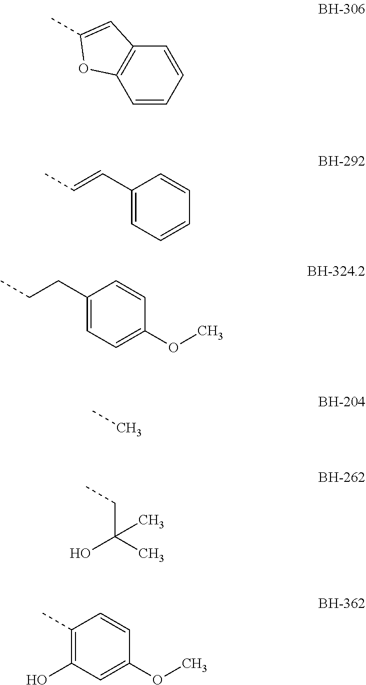

12. Compound according to claim 1, wherein the protozoa have the nitrite-formiate-transporter protein of Plasmodium falciparum, of Plasmodium vivax, of Toxoplasma gondii or of Entamoeba histolytica or the Gly107Ser mutant thereof.
